(19) United States
(12) Patent Application Publication
(10) Pub. No.: US 2009/0221888 A1
(43) Pub. Date: Sep. 3, 2009

(54) WEARABLE SENSOR SYSTEM FOR ENVIRONMENTAL AND PHYSIOLOGICAL INFORMATION MONITORING AND INFORMATION FEEDBACK SYSTEM

(76) Inventor: RAVINDRA WIJESIRIWARDANA, Bentonville, AR (US)

Correspondence Address: RAVINDRA WIJESIRIWARDANA, 2001 LATROBE DRIVE APT 20, BENOTVILLE, AR, 72712, US

(21) Appl. No.: 12/395,771

(22) Filed: Mar. 2, 2009

Related U.S. Application Data
(60) Provisional application No. 61/033,132, filed on Mar. 3, 2008.

(51) Int. Cl. A61B 5/1455; A61B 5/01; A61B 5/08; A61B 5/083

(52) U.S. Cl. 600/324; 600/549; 600/529; 600/532

(57) ABSTRACT

An ear wearable or head wearable environmental information and physiological information monitoring device is introduced. This system is capable of transmitting and receiving information wirelessly. This system also comprises of a wireless display unit that can be attached to a hand band.

### CROSS-REFERENCE TO RELATED APPLICATIONS

This application claims the benefit of provisional patent application Ser. No. 61/033,132, filed Mar. 3, 2008 by the present inventor.

### FEDERALLY SPONSORED RESEARCH Not Applicable

### SEQUENCE LISTING OR PROGRAM

Not Applicable

### BACKGROUND

1. Field

This application relates to wearable physiological information and environmental information monitoring systems.

2. Prior Art

Head wearable and ear wearable physiological information monitoring systems are getting popular due to their advantages, including easiness in wearing, the improvement in accuracy and reliability of the information. The head or ear mounted devices use pulse plethysmography (PPG) or oximetry (SpO₂) to monitor the heart rate. The body surface temperature is measured by using an electrical thermometer mounted on the skin of the wearer. The respiration information is measured by using optical inductive capacitive, resistive or electrical plethysmography in these wearable systems.

### SUMMERY OF THE PRESENT INVENTION

The present invention discloses an ear wearable wireless mammalian body temperature and environment temperature measuring unit. Also the present invention discusses an ear or head wearable PPG based wireless heart rate monitoring system, an ear wearable or head wearable respiration monitoring system based on the temperature difference of the inhale and exhale air, or concentration difference of the carbon dioxide and carbon monoxide of the inhale and exhale air, or the humidity difference between the inhale and exhale air. In addition this respiration information monitoring system can be integrated into head wearable gear such as caps, helmets and head bands. Also the present invention includes a wearable wireless display unit that is capable of displaying the physiological and environmental information. The display unit is capable of connecting to a wrist worn watch or a body worn band or strap. In addition this system is capable of transmitting, recording and displaying information in real time.

### DRAWING—FIGURES

FIG. 1A—Ear wearable wireless thermometer with an ear canal insert thermal sensor.

FIG. 1B—Ear wearable wireless thermometer with an ear canal insert thermal sensor and a information display screen.

FIG. 1C—Wearable wireless thermometer with an attachable display unit and either trans virginal or ear canal insert temperature probe.

FIG. 1D—Shows the time response of the temperature measured with an ear wearable wireless thermometer.

FIG. 2A—Ear wearable wireless respiration information measuring device based on temperature sensors.

FIG. 2B—The sensor unit with thermal sensors and the conduction path ways.

FIG. 2C—A person wearing the ear wearable respiration information monitoring device based on thermal sensors.

FIG. 2D—shows the thermal sensor placement.

FIG. 3A—Shows the head wearable wireless temperature, heart rate, respiration information measuring system.

FIG. 3B—Shows a person wearing a device described in FIG. 3A.

FIG. 3C—Shows an ear wearable wireless temperature, heart rate, respiration information measuring system.

FIG. 3D—Shows a temperature sensors based respiration information monitoring helmet or head gear.

FIG. 3E—Shows the respiration signal picked up by the sensors.

FIG. 4A—Shows the wearable wireless display unit that can be attached to a belt of a wrist watch or belt or a strap worn by a wearer.

FIG. 4B—Show the device of FIG. 4A clipped or attached to a wrist worn watch.

### DRAWINGS—REFERENCE NUMERALS

001—Ear hook.
002—Ear canal insert thermometer.
003—Wearable wireless thermometer.
004—Mammalian ear.
005—Temperature information display screen
006—Control switch
010—Temperature probe
007—Conduction pathways between the display unit and the temperature probe.
008—Wireless and attachable temperature information display unit.
011—Nose clip with thermal sensors.
012—Substrate of the mouth positioned thermal sensors
013—Thermal sensor.
018—Connector of the 011 and 012 parts
015—Ear wearable wireless signal conditioning unit.
016—Ear hook of the 015
017—Ear plug of the 015
014—Control switch of the 015
009—Conduction pathways between the sensory module and the wireless signal conditioning unit.
019—Mouth
020—Nose
021—Head band
022—Pressure pad.
024—Wireless signal conditioning unit
025—Temperature ear probe.
029—PPG sensor either clippable to the ear penne or attachable to the skin of the wearer
028—Connector between the sensors and sensor holder.
027—Thermal sensors.
023—Connector cable between the PPG sensor and the wireless signal conditioning unit.
030—Ear hook
031—Helmet
033—Wireless signal condoning unit
032—Connector cable between the thermal sensors and the signal conditioning unit (033)
037—Connector clip or the belt
038—Control switches
036—Screen
034—Loop hole for the belt
035—Casing of the display unit
040—Arm of a person.
039—Wrist worn strap belt or a watch.

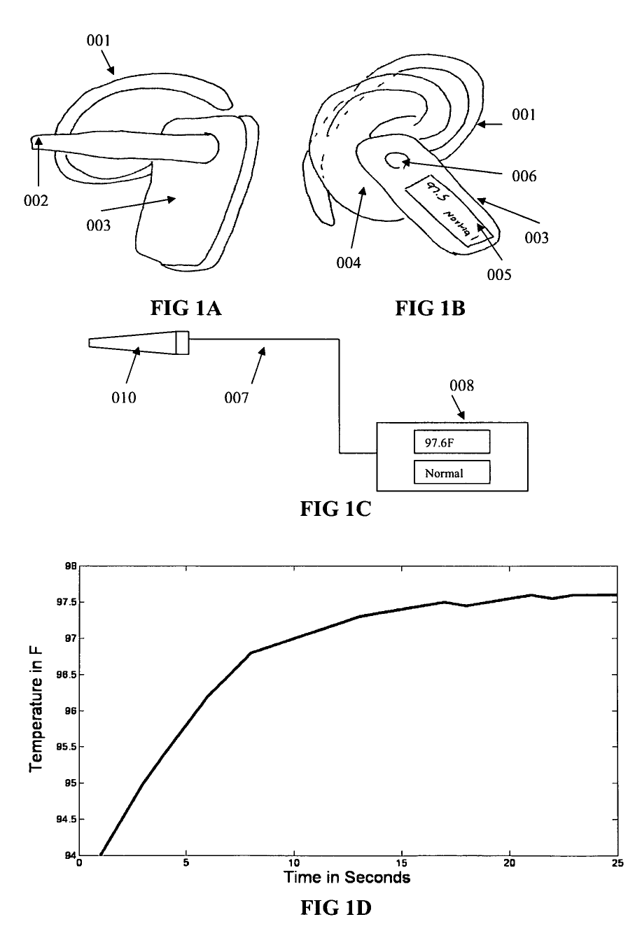

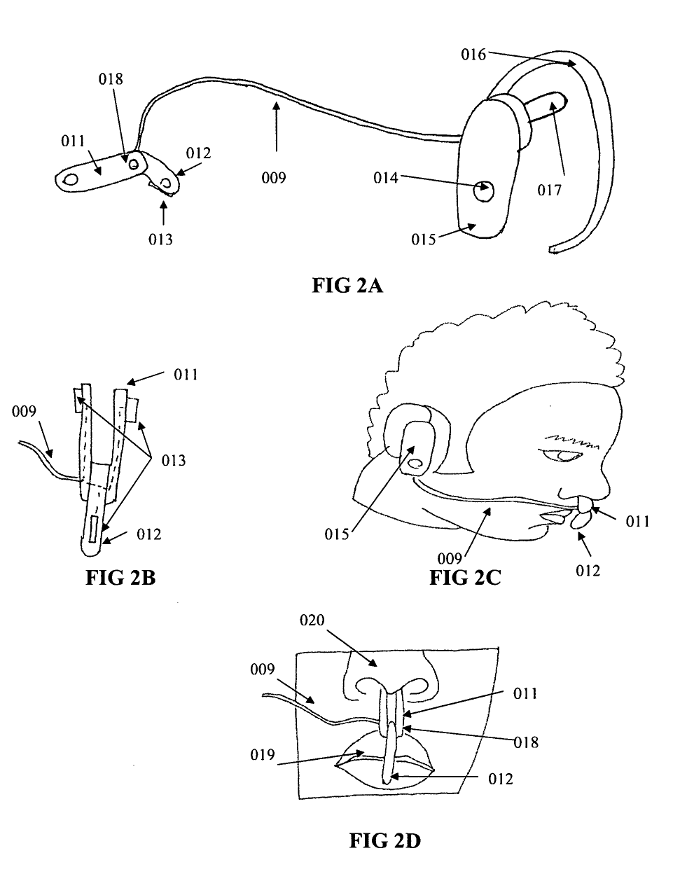

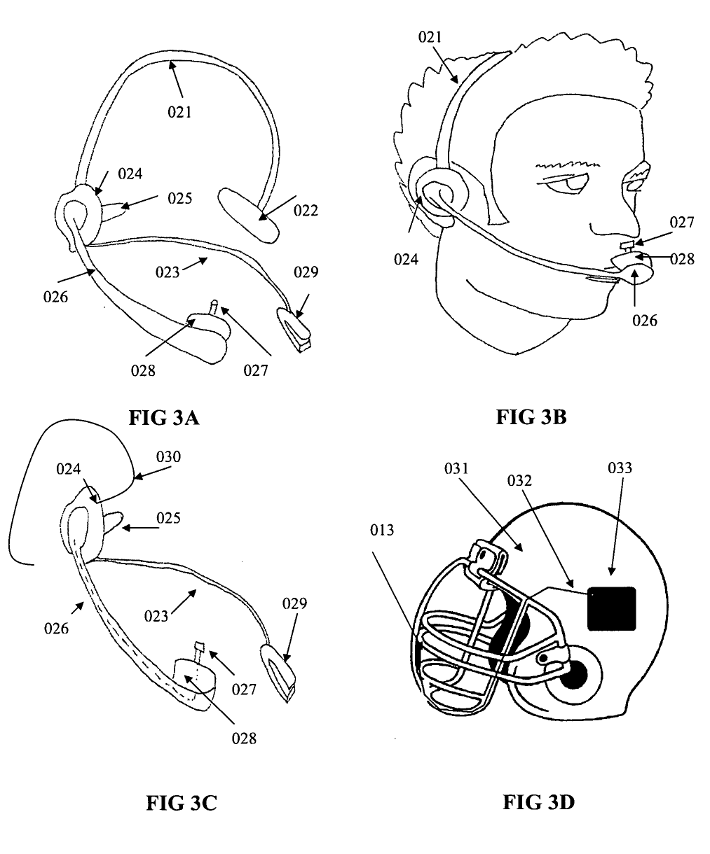

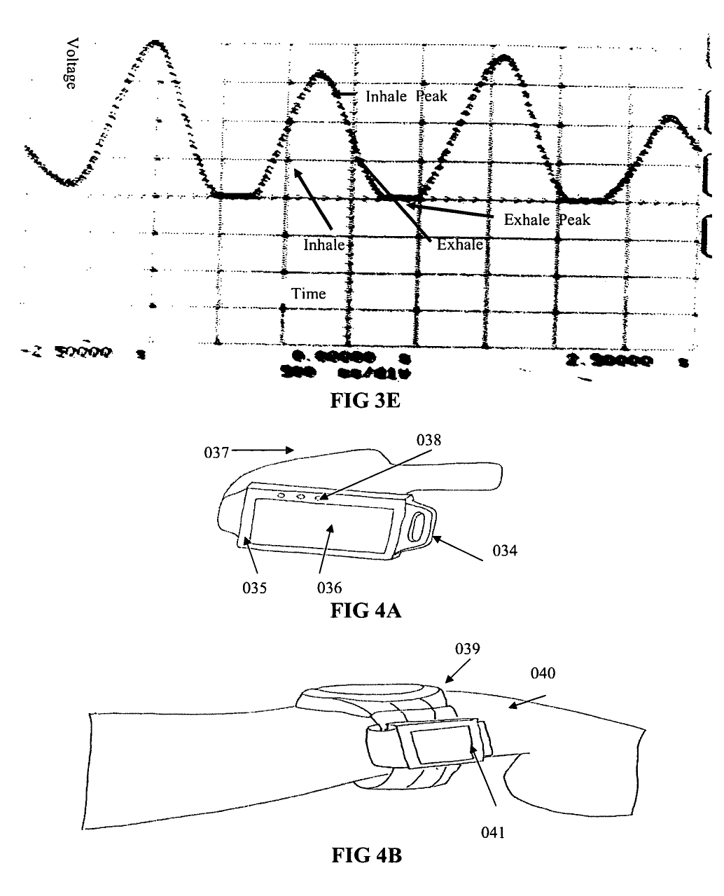

DETAILED DESCRIPTION OF FIG. 1A, FIG. 1B, FIG. 1C, FIG. 1D, FIG. 2A, FIG. 2B, FIG. 2C, FIG. 2D, FIG. 3A, FIG. 3B, FIG. 3C, FIG. 3D, FIG. 3E, FIG. 43A and FIG. 4B FIG. 1A and FIG. 1B show an ear wearable wireless temperature monitoring unit with an ear canal temperature probe (002). The Probe (002) is connected to the casing of the signal conditioning unit. The device also has an ear hook to reduce the motion artifacts of the system. In addition the device consists of thermal information displaying unit. This display is used for the temperature display and messaging. This unit may contain an audio feed back unit to communicate with the wearer.

FIG. 1C shows an attachable temperature monitoring device with either trans virginal or ear canal insert temperature probe. This device is also capable of wireless signal transmission. This device can be attached to the wearer garment via a clip.

FIG. 1D shows the temperature distribution with time of the ear wearable wireless temperature monitor discussed in FIG. 1A.

FIG. 2A shows an ear wearable wireless respiration information monitoring device based on thermal sensors. The thermal sensors in the sensor unit are connected to the signal conditioning unit (015) via a signal and power transmitting cable (009). The casing of the signal conditioning unit has an ear plug (017) and an ear hook for the better connection of the unit to the body. FIG. 2B shows the thermal sensor unit having nose clip (011) with thermal sensors (013) and a substrate to hold mouth thermal sensor. The angle between the 011 and 012 can be adjusted at the 011 and 012 joining point (018).

FIG. 3A and FIG. 3B shows a head wearable multiple physiological parameter monitoring system. This device contains an ear canal invertible temperature probe connected to the housing of the signal conditioning unit, a PPG sensor/s for the hear rate monitoring and thermal sensors based respiration information measuring unit. FIG. 3C show the same device with ear wearable configuration with a hook (030). FIG. 3D shows a thermal sensor based respiration information monitoring system integrated with a helmet. The thermal sensors (013) are on front rack of the helmet.

FIG. 3E shows the voltage vs. time graph of the thermal sensors. The respiration information can be obtained very accurately by the temperature variations.

FIG. 4A shows a strap wrist band attachable wireless display unit for physiological information and environmental information displaying. FIG. 4B shows this device attached to a wrist watch.

### OPERATIONS OF THE INVENTION

FIG. 1A and FIG. 1B show an ear wearable temperature monitoring device having an ear canal insert temperature sensor. The ear canal sensor detects the body core temperature and sends it to the ear wearable signal conditioning unit where the signal is filtered and processed. Then this signal is transmitted to the external monitoring station. The external monitoring station may be a body worn display unit, one described in FIG. 4A, FIG. 4B or a mobile phone or a personal digital assistant (PDA) or an external display unit. FIG. 1C is a wireless temperature monitoring unit that can be attached to a wearer. The attachments can be made to the wearable garment of the wearer. The attachment can be achieved via clips or snaps. This unit consists of a temperature probe. The device can be used to monitor the temperature variation of a female during the ovulation time and inform the best time for the conception. FIG. 1D shows a typical temperature output of the ear wearable wireless thermal unit.

FIG. 2A shows an ear wearable respiration information monitoring system where the respiration information is monitored via a sensor unit near the vicinity of the nose and the mouth of the wearer. This sensor unit (FIG. 2B) comprises of temperature sensors or humidity sensors or air flow sensors or carbon dioxide and carbon monoxide sensors or ammonia sensors.

The sensor unit comprises of nose clip (011) with sensor (013) and substrate (012) to hold the sensors for the mouth area. The sensors of this unit pick up the temperature variation or humidity variation, or carbon dioxide variation or carbon monoxide variation of the inhale and exhale air and transmit the signal to the ear wearable wireless signal conditioning unit. Then this signal is transmitted to the external monitoring device wirelessly.

FIG. 3A shows a head wearable wireless physiological information monitoring unit having PPG heart rate monitoring device, temperature based respiration information monitoring unit and a ear canal insertable temperature monitoring device. This device can be incorporated into an ear wearable arrangement (FIG. 3C) or a head gear arrangement (FIG. 3D) such as a helmet cap head band.

FIG. 3E shows the voltage vs time graph of the temperature variation picked up by the sensor unit during a person inhale and exhale.

The wearable display unit is capable of displaying physiological information of the wearer. The physiological information may be sent to this by an electrocardiogram monitoring unit, or a heart rate monitoring unit, or a respiration information monitoring unit, or a temperature monitoring unit.

I claim:

1. Wearable body core temperature measuring system with an ear canal insert type thermal sensors or trans-virginal temperature sensors having a signal processing, signal conditioning, information feed back, control signal feed back, power supply and display units are in an ear wearable embodiment.

2. A device according to claim 1 having a signal processing unit, signal feed back unit, and display unit are in the same embodiment/s that cam be clipped to user wearable garment such as a cap, a hat, a head band, a shirt, an underwear, a jacket or trouser.

3. A device according to the claim 1 or claim 2 having an external base station such as a computer (PC), PDA, mobile phone, wrist worn watch or a wearable display unit for monitoring, processing, displaying and generating alarms and generating audio or visual feed back to the wearer.

4. A devices according claim 1 or claim 2 or claim 8 or claim 9 having a signal feed back unit comprises of audio feedback speaker.

5. Use of a device according to claim 1 or claim 2 or claim 3 to monitor basal temperature of women and providing the feed back for monthly ovulation cycle and best time for conception.

6. The audio visual feed back to the wearer is achieved by the means of temperature information and the level of the temperature stating normal, low or high.

7. A device according to the above claim 1 or claim 2 or claim 3 or claim 4 or claim 5 having a memory so that the temperature can be stored and downloaded to an external device such as a PDA or a PC.

8. An ear wearable or a head wearable respiration information monitoring system that measures the respiration information of a mammal via the temperature change of the inhale and exhale air, or the temperature difference of the surface of the lips or the mouth or inner or outer surface of the nose of a mammal, or the humidity variation of the inhale and exhale air, or the gas concentrations variation of the inhale and exhale air.

9. A system according to claims 8 having a wire or wireless information transmission unit that transmits the respiration information including but not limited to breath temperature, inhaling duration, inhaling dept, inhaling gas concentrations, exhaling duration, exhaling depth, exhaling gas concentrations, mouth breathing, mouth breathing duration and depth, nasal breathing depth, nasal breathing duration, respiration rate (number of breadths per minute), breath flow rate and humidity of the inhale and exhale air to the display, recording, processing unit.

10. The gas concentrations variation of the inhale and exhale air of the claim 8 is carbon dioxide or carbon monoxide or oxygen or gases with nitrogen compound.

11. A sensor system according to claim 8 having a display unit or audio unit for the audio visual feedback information to the wearer.

12. A system according to claim 8 where the system can be attached onto or embedded into wearable apparatus including; a helmet, a cap, a head band, an ear wearable device, a head wearable goggles, a jacket, a garment, a face mask, a balaclava, an air breathing apparatus of a pilot, an under water diver snorkeling gear, a swimming cap or an optical spectacles.

13. The sensor unit of a device according to the claim 8 or claim 9 having a reusable or disposable sensor unit that can be attached to the surface of the face of a mammal via a nose clip or a mouth lip clip or by using adhesives or by any combination of these.

14. The thermal sensors of a device according to claim 8 or claim 9 for measuring the respiration information may be thermocouples, thermistors, thermopile sensors, PZT sensors, semiconductor thermal sensors or any combination of these.

15. A wearable device according to claim 8 or claim 9 with a thermal PPG Sensor/SpO₂ sensor integrated, embedded or attached together with respiration information monitoring sensor unit to the same device so that the heart rate can be picked up by the same sensor unit.

16. A device according to claim 8 or claim 9 with the device stands on the head or on a one ear of the wearer and PPG/SpO₂ sensors attached to the other ear and the PPG/SpO₂ sensors are connected to the device via a cable carrying the signals and power conductive pathways.

17. A device according to claim 1 or claim 2 or claim 3 or claim 8 or claim 9 having additional environmental information monitoring sensors such as air quality, radiation, geographical location, position, direction, temperature and humidity.

18. A system comprises of a device according to claim 1 or claim 2 or claim 3 or claim 8 or claim 9 and a wireless environmental information monitoring device such as air quality, radiation, geographical location, position, direction, temperature and humidity.

19. A device according to claim 8 or claim 9 or claim 1 or claim 2 or claim 3 having microphone for audio communication and a ear canal insert temperature sensor for the body core temperature measurement.

20. Use of a device according to claim 8 or claim 9 or claim 15 or claim 16 or claim 17 or claim 18 or claim 19 to identify early development of breathing difficulty, an asthma attack and dehydration of a person.

21. A hand band or a wrist watches band attachable wireless display unit for displaying physiological information and environmental information. This unit is either clipped or snapped to the band or strapped around the band with a Velcro adjustable band.
